(19) United States
(12) Patent Application Publication
(10) Pub. No.: US 2009/0068112 A1
(43) Pub. Date: Mar. 12, 2009

(54) Magnetic Nanoparticles for Imaging

(76) Inventor: Yousef Haik, Greensboro, NC (US)

Correspondence Address: AKERMAN SENTERFITT, P.O. BOX 3188, WEST PALM BEACH, FL, 33402-3188, US

(21) Appl. No.: 12/205,601

(22) Filed: Sep. 5, 2008

Related U.S. Application Data
(60) Provisional application No. 60/971,286, filed on Sep. 11, 2007.
(60) Provisional application No. 61/027,449, filed on Feb. 9, 2008.

(51) Int. Cl. A61K 49/06

(52) U.S. Cl. 424/9.3

(57) ABSTRACT

A medical imaging system that enables the discovery of malignant tissue utilizing contrast agents and heating agents made of magnetic nanoparticles that are delivered to tumor sites utilizing attenuated strains of bacteria that seek and reside at tumor sites is disclosed. The thermal contrast agents may be temperature self-controlled magnetic nanoparticles that may be encapsulated in a biocompatible coating. The thermal contrast agents may be uploaded into attenuated strains of bacteria that seek and reside in tumor tissue when placed into a bloodstream of a patient. An alternating magnetic field device with a prescribed frequency range may be used to induce heating of the magnetic nanoparticles in the patient, and a thermal scan may be utilized to identify tumors. In another embodiment, the contrast agent may be formed from magnetic nanoparticles having distinct magnetic moment profiles, and a MRI system may be utilized to identify tumors with such contrast agent.

### CROSS-REFERENCE TO RELATED APPLICATION

This patent application claims the benefit of U.S. Provisional Patent Application No. 60/971,286, filed Sep. 11, 2007 and claims the benefit of U.S. Provisional Patent Application No. 61/027,449, filed Feb. 9, 2008, both of which are incorporated by reference.

### FIELD OF THE INVENTION

This invention is directed to a medical imaging system, and more particularly, to a medical imaging system that enables the discovery and diagnosis of malignant tissue utilizing contrast agents and heating agents made of magnetic nanoparticles that are delivered to tumor sites utilizing attenuated strains of bacteria that seek and reside at tumor sites.

### BACKGROUND OF THE INVENTION

Malignant tumors are the second leading cause of death in the United States after the heart disease. Abnormal or neoplastic cells proliferate to form tumors, invade normal tissue and generate free malignant cells that spread through blood streams of patients. Therapeutic schemes depend on the accurate diagnosis of the level of pathological grading of the invasiveness and aggressiveness of the cancer manifestation.

Robust advances in medical imaging have occurred in recent years. Utilization of magnetic material has emerged as MRI contrast agents in whole body imaging and localization of clinically important physiological sites such as angiogenesis, oxygen insufficiency and ischemia. Noninvasive diagnostic techniques are preferred for routine check-ups and early diagnosis. Magnetic resonance imaging (MRI) technology has grown over the recent years to become one of the most practiced techniques for imaging. MRI systems are capable of showing different characteristics of the imaged tissue by identifying the different levels of magnetization of the tissue subjected to a specific signal. MRI systems provide more data than that of CT and ultrasound. MRI systems are useful to locate tumor foci. The levels of magnetization of the various tissue are displayed as different contrasts. The MRI systems enable accurate characterization of pathological areas with high resolution.

Different types of magnetic particles can be used as contrast agents to enhance the MRI signal. For example, iron oxide particles have been used as contrast agents because the iron particles shorten the effective transverse relaxation time (T2) of tissues that take up these particles. Gadolinium diethyltriamine pentaacetic acid (Gd-DTPA) has been also used as a contrast agent that primarily shortens longitudinal relaxation time (T1) resulting in intensity enhancement. However, most conventional contrast agents have poor specificity.

Currently, coated or noncoated contrast agents are placed into the bloodstream of a patient, whereby the tumor feed mechanism attracts the contrast agents to the tumor site. In this system, both normal and tumor tissue have contrast agents attached thereto, thereby making identification of tumors difficult. MRI software reports the imaging results, however, there are several other problems in delivering the tagged contrast agents to a particular site. First, contrast agents need to have a proper tag that will facilitate delivery from the work site. Second, a conventional contrast agent is Gadolinium Diethylene-Triaminepentaacetic Acid (GD-DTPA), magnetic particles, and bubbled spheres. Gadolinium is a toxic agent when present by itself.

Conventional technology has used a direct current magnetic field to draw magnetic contrast agents to a tumor site, as set forth in U.S. Pat. No. 6,514,418. However, a major disadvantage of this technology is the need to concentrate high steady fields at the tumor site, such as a magnetic field of seven tesla as set forth in the '418 patent, to attract magnetic carriers to the tumor. The cost, size and cooling requirements of a magnet capable of generating such a magnetic filed hinder the utilization of such fields in clinical settings. Additionally, the effects of a continuous seven tesla magnet on normal tissue is unknown.

### SUMMARY OF THE INVENTION

This invention is directed to a contrast agent configured to readily enter and reside in a tumor within a living being, such as a human or animal, so that the tumor may be identified with thermal imaging and MRI systems. The contrast agent may include active attenuated strains of bacteria that have the ability to seek and reside at tumor sites. Utilizing attenuated bacteria as a carrier overcomes the issues associated with tagging and delivering conventional contrast agents to a site of interest such as a tumor. Genetically modified strains of bacteria such as, but not limited to, Salmonella typhimurium, accumulate at tumor sites when injected in tumor-bearing mice and clear rapidly from blood in normal mice. The innovative delivery system makes use of genetically modified strains of bacteria, which includes genetically stable attenuated virulence (deletion of purl gene), reduction of septic shock potential (deletion of msbB gene) and antibiotic susceptibility.

In one embodiment, the contrast agent for imaging may be at least one magnetic nanoparticle having a Curie temperature less than a critical temperature of tissue at which the tissue is compromised. The magnetic nanoparticle may include additional functions, such as, but not limited to, acting as a hyperthermia agent. The Curie temperature of the magnetic nanoparticle may be between about 40° C. and about 44° C. The contrast agent for imaging may also be at least one magnetic nanoparticle having a distinct magnetic moment profile that coorelates with specific temperatures and is detectable with MRI systems.

These contrast agents may be delivered to tumors within a patient with a delivery system in which at least one magnetic nanoparticle is in contact with an attenuated, nonpathogenic bacteria strain. In particular, the magnetic nanoparticles may be uploaded to attenuated bacteria strains. The predetermined concentration of bacteria may be placed, through injection or otherwise, into a bloodstream of a patient, such as a human being or animal, to identify tumors within the patient. Once in the bloodstream, the bacteria seeks the tumor. Once the bacteria locates the tumor, the bacteria enters the tumor and resides therein. If no tumor is present, the attenuated bacteria strains are passed out of the patient within 24 hours of being injected into the patient. An alternating magnetic field may then be applied in proximity of the tumor location about 24 hours after administering the loaded bacteria to the patient. The magnetic nanoparticles induce heating within the tumor tissue. In embodiments where the contrast agents are those having a Curie temperature less than a critical temperature of tissue at which the tissue is compromised, the magnetic nanoparticles, when subjected to the alternating magnetic field, heat up to a predetermined Curie temperature and do not increase in temperature beyond the Curie temperature. A thermal sensitive device is utilized to scan the patient's body to identify the thermal signature of the patient. The contrast agents attached to the attenuated bacteria strains collect in the tumors throughout the patient and increase in temperature to temperatures greater than other parts of the patient. Thus, the location of tumors within the patient are identified by identifying high temperature spots within the patient. In embodiments in which contrast agents with distinct magnetic moment profiles are used, a MRI system may be used to identify the tumor.

An advantage with this invention is that the contrast agents may be attached to bacteria strains that are readily taken up by tumors, thereby forming a delivery mechanism that effectively delivers that contrast agents to a tumor such that the tumor may be easily identified by a concentration of contrast agents therein.

Another advantage of this invention is that the contrast agents heat up due to exposure to an alternating magnetic current to a temperature below a temperature at which damage can occur to surrounding tissue.

Yet another advantage of this invention is that the contrast agents attached to attenuated bacteria strains will also assist in detecting small size tumors that cannot otherwise be discovered.

Another advantage of this invention is that the delivery system reduces the issues associated with specificity to tumor detection.

Still another advantage of this invention is that the delivery system reduces the risk of inducing a toxic reaction at normal sites because the delivery system does not use toxic materials, which were used in conventional systems.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 depicts a transmission electron microscopy image of the coated magnetic nanoparticles that can be utilized as thermal and or magnetic contrast agents.

FIG. 2 is an image of targeted sites in a human body with magnetic nanoparticles.

FIG. 3 depicts experimental results for magnetic nanoparticles that were excited with an alternating magnetic filed at a frequency of 963 MHz and stopped heating at the Curie temperature for the magnetic nanoparticles.

FIG. 4 depicts a top view of tubes filled with agarose gel doped with different concentrations of magnetic nanoparticles, wherein the tubes are surrounded by a water phantom to maintain temperature.

FIG. 5A depicts T1 relaxivity (R₁ in s⁻¹) showing a linear increase with concentration at 39 degrees Celsius.

FIG. 5B depicts T2 relaxivity (R₂ in s⁻¹) showing a linear increase with concentration at 39 degrees Celsius, whereby the increase in R2 is relatively larger than the increase in R1.

FIGS. 6A-C are TEM micrographs for three different FNB compositions that are referred to as (A) TEM for FNB1, (B) TEM for FNB2 and (C) TEM for FNB3.

FIG. 7 is a photograph of silica coated particles.

FIG. 8 is graph of an applied magnetic field vs. magnetization plot.

FIG. 9A is an image of encapsulated impregnated magnetic nanparticles attached on the cell membrane of Salmonella.

FIG. 9B is an image that depicts smaller magnetic nanoparticles having entered the Salmonella's membrane.

FIG. 9C is an image depicting vertically aligned Salmonella when exposed to a magnetic field placed on top of the slides.

FIG. 10 is a graph of the uploading process of the magnetic nanoparticles to the bacteria.

FIG. 11 is a comparison of photographs of different concentrations of cytotoxicity of fibroblast and caco-2.

FIG. 12 is an image depicting a temperature map of a heated tumor containing magnetic nanoparticles.

### DETAILED DESCRIPTION OF THE INVENTION

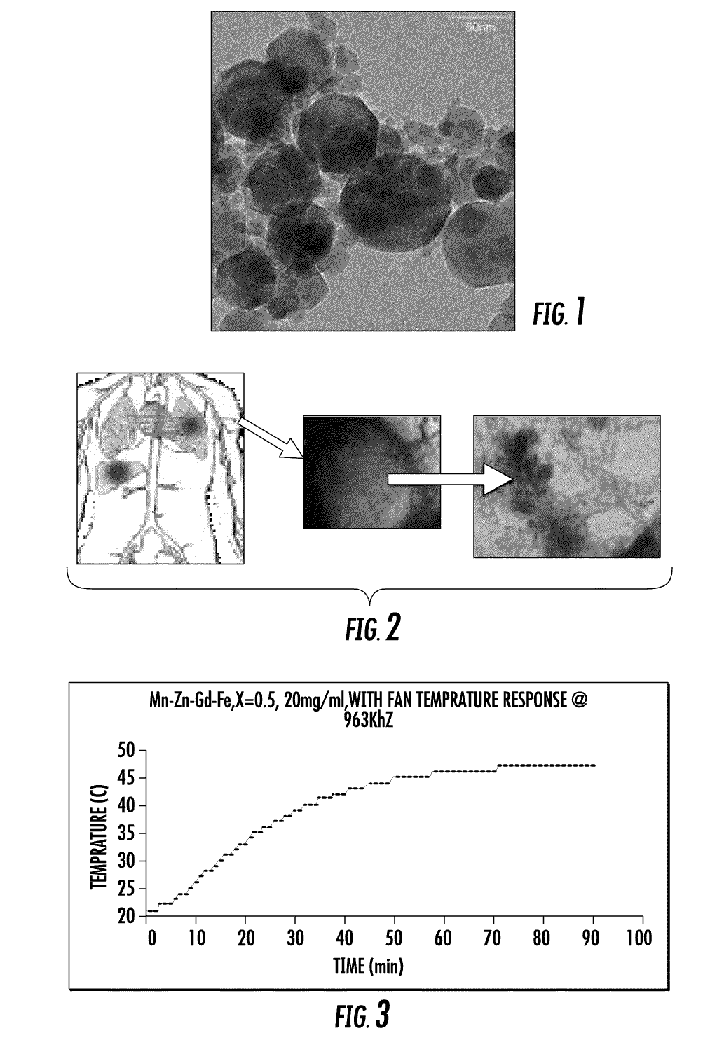

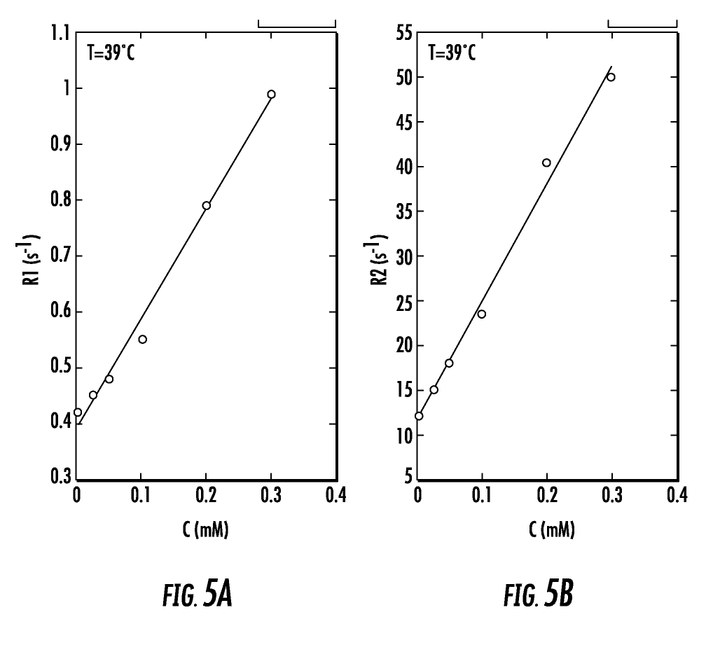

As shown in FIGS. 1-12, the invention is directed to a contrast agent configured to readily enter and reside in a tumor within a living being, such as a human or animal, so that the tumor may be identified with thermal imaging and MRI systems. The living being may be referred to as a patient herein. The contrast agent may include active attenuated strains of bacteria that have the ability to seek and reside at tumor sites. Utilizing attenuated bacteria as a carrier overcomes the issues associated with tagging and delivering conventional contrast agents to a site of interest in a patient.

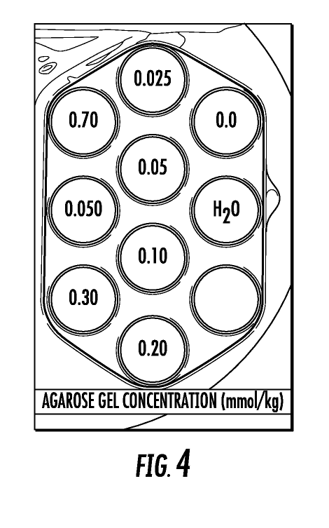

In one embodiment, the contrast agent may be formed from one or more magnetic nanoparticles, as shown in FIG. 1. In another embodiment, the magnetic nanoparticles may have a Curie temperature less than a critical temperature of tissue at which the tissue is compromised such that the contrast agent may be heated to a temperature not to exceed the Curie temperature, thereby preventing damage to the surrounding tissue, as shown in FIG. 3. A delivery system for a contrast agent for use in imaging systems is disclosed in which the magnetic nanoparticle having a Curie temperature less than a critical temperature of the tissue is placed in contact with a carrier that is an attenuated bacteria strain to facilitate uptake of the contrast agent by a tumor, as shown in FIG. 2. In another embodiment, the contrast agent may be at least one magnetic nanoparticle having a distinct magnetic moment profile with particular temperatures. A delivery system for a contrast agent for use in imaging systems is disclosed in which the magnetic nanoparticle having a distinct magnetic moment profile with particular temperatures is placed in contact with a carrier that is an attenuated bacteria strain to facilitate uptake of the contrast agent by a tumor.

The magnetic nanoparticles may be formed from particles having cross-sections between about five nm to less than one micron in width. In other embodiment, the magnetic nanoparticles may be larger particles. As used herein, the term “magnetic nanoparticles” includes magnetic, paramagnetic, superparamagnetic ferromagnetic and ferrimagnetic materials. The nanoparticles may be formed from a combination of magnetic and nonmagnetic materials. Such combinations may be configured to have a Curie temperature between about 40° C. and about 44° C., as shown in FIG. 3. As such, when the magnetic nanoparticles are excited by an alternating magnetic field, the magnetic nanoparticles experience a temperature rise up to, but not exceeding, their Curie temperature. The magnetic nanoparticles may be synthesized using chemical or physical methods. For instance, the magnetic nanoparticles may be formed from NiCu, NiCr (binary magnetic nanoparticles), MnGdFe, ZnGdFe, FeGdB, FeNdB (tri magnetic nanoparticles), and MnZnGdFe (quad magnetic nanoparticles).

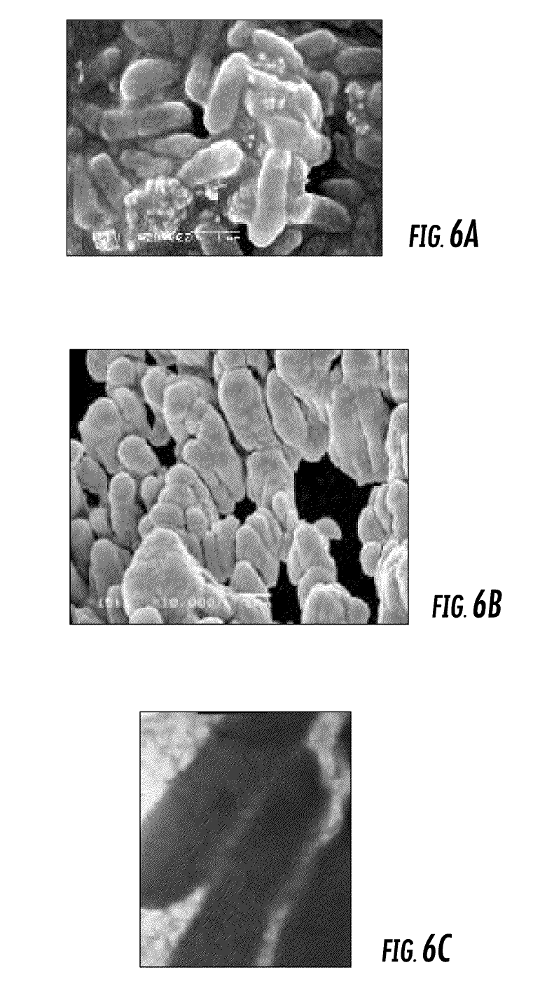

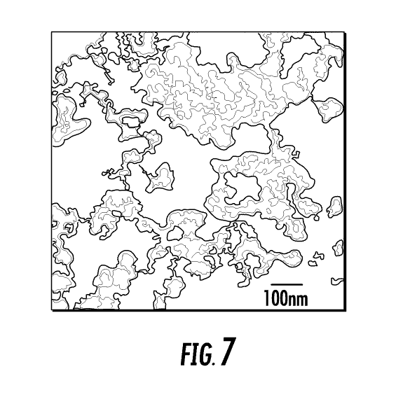

The magnetic nanoparticles may be formed using co-precipitation method and borohydride reduction method. For example, FeGdB and FeNdB nanoparticles have been synthesized using a borohydride reduction method. Ultrasonication was used to form homogeneously sized particles in the nanometer range (30-100 nm). In order to minimize the oxidation effect, these particles were passivated overnight under a continuous flow of oxygen and nitrogen. These particles were further coated with silica by using a precipitation process and have been designed for biomedical applications. Silica coated particles are capable of chemically attaching to proteins and many other biological molecules. Morphology and sizes of all of the magnetic nanoparticles were determined with scanning transmission electron microscopy (TEM). The magnetic properties of the magnetic nanoparticles were studied using a Superconducting Quantum Interference Device (SQUID) magnetometer. The composition of the alloy material was determined by inductively coupled plasma atomic emission spectroscopy (ICP). The ICP analysis of magnetic nanoparticles identified the stoicheometric composition of three alloys as Fe_{5.6}NdB₂, Fe_{5.4}NdB₉ and Fe_{5.2}NdB_{13.7}. For the purpose of discussion, the samples are designated as FNB1, FNB2 and FNB3 respectively. The composition of the alloy is strongly dependent on the reaction conditions. FIGS. 6A, 6B and 6C show the TEM micrograph for FNB 1, FNB2 and FNB 3, respectively. The magnetic nanoparticles are mostly in the size range of between about 50 nm and about 200 nm. FIG. 7 shows an image of silica coated FeNdB particles. The micrograph depicts that the cluster of the magnetic nanoparticles rather than individual particles have been coated with silica. The hysterics plots shown in FIG. 8 at 300K indicate the superparamagnetic nature of all samples as there was almost no remnant magnetization. In addition, control of the composition of the elements during the reaction is necessary in order to control the magnetic moment, and hence to, tune the magnetic nanoparticles for superior imaging contrast properties. Other contrast agents include iron oxide particles prepared by a co-precipitation method. The iron oxide particles may have a narrow size distribution (2-10 nm). These iron oxide particles were then functionalized to be used as a contrast agent for mouse heart tissue by coating the iron oxide particles with anti-myglobin antibodies.

FIG. 4 depicts a MRI image for different concentrations of MnGdFe particles suspended in agrose gel. The MRI image shows an enhancement of the signal as the concentration increases. The concentrations used in this experiment are within the therapeutic range of conventional contrast agent limits when injected into a human.

In at least one embodiment, the magnetic nanoparticles may be encapsulated by one or more biocompatible coatings. The encapsulated contrast agents may have a cross-section between about 10 nm and 1 micron. The biocompatible coating may be, but is not limited to, a polymeric material, a biodegradable material, and a protein. A polymeric material may be, but is not limited to, one or more oligomers, polymers, copolymers, or blends thereof. Examples of polymers include polyvinyl alcohol; polyethylene glycol; ethyl cellulose; polyolefins; polyesters; nonpeptide polyamines; polyamides; polycarbonates; polyalkenes; polyvinyl ethers; polyglycolides; cellulose ethers; polyvinyl halides; polyhydroxyalkanoates; polyanhydrides; polystyrenes; polyacrylates, polymethacrylates; polyurethanes; polypropylene; polybutylene terephthalate; polyethylene terephthalate; nylon 6; nylon 6,6; nylon 4,6; nylon 12; phenolic resins; urea resins; epoxy resins; silicone polymers; polycarbonates; polyethylene vinylacetate; polyethylene ethyl acrylate; polylactic acid; polysaccharides; polytetrafluoroethylene; polysulfones and copolymers and blends thereof. The polymeric material may be biocompatible and may be biodegradable. Examples of suitable polymers include ethylcelluloses, polystyrenes, poly(ε-caprolactone), poly(d,l-lactic acid), polysaccharides, and poly(d,l-lactic acid-co-glycolic acid). The polymer may be a copolymer of lactic acid and glycolic acid (e.g., PLGA). The protein may be, but is not limited to, BSA or HSA.

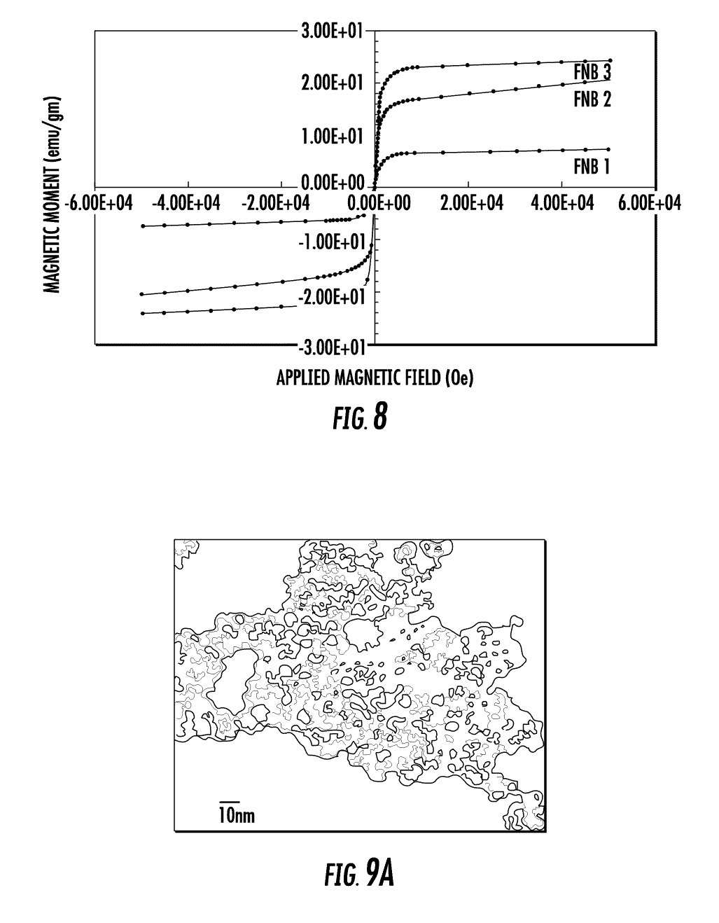

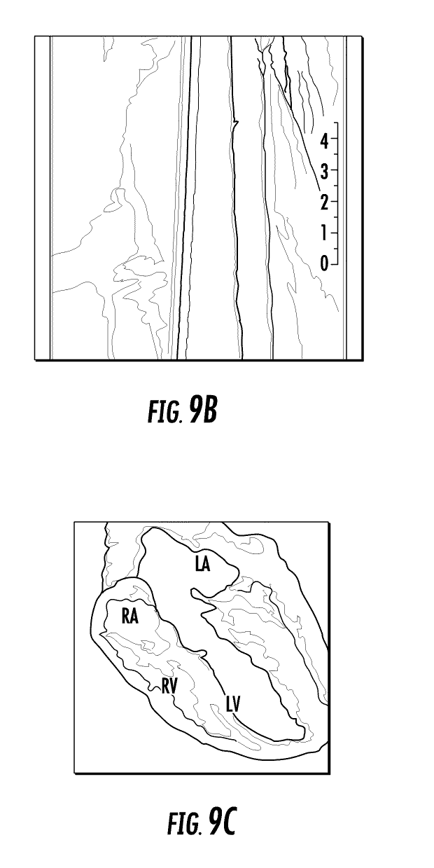

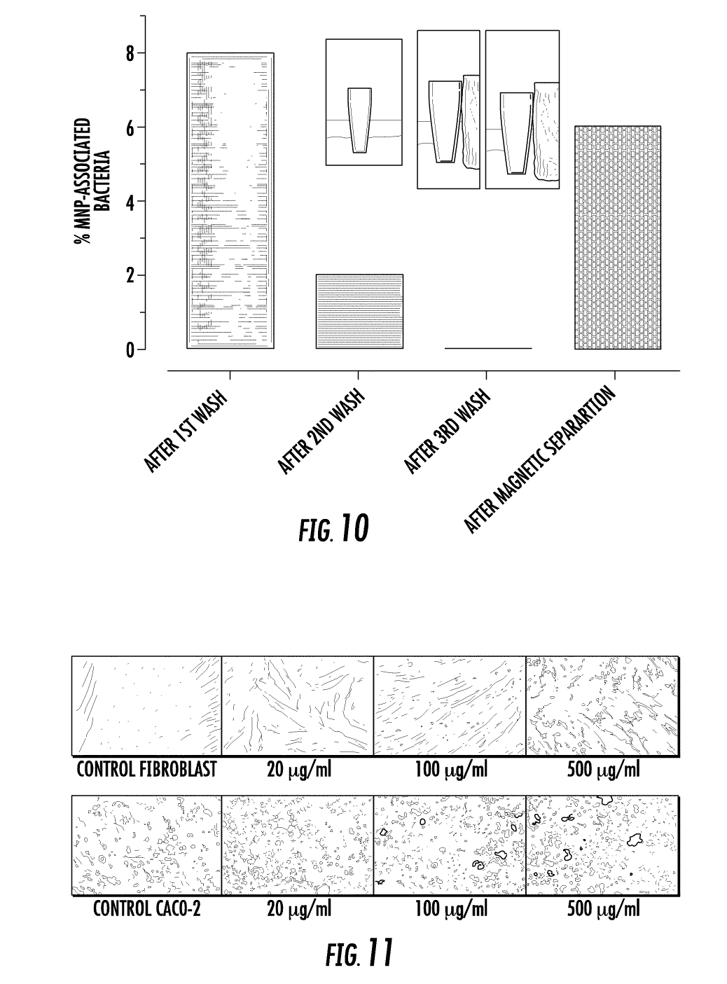

In one embodiment, the contrast agents may be uploaded to attenuated bacteria strains to facilitate greater uptake by a tumor of the contrast agents, as shown in FIG. 9. The attenuated bacteria strains may be genetically modified strains of bacteria, including genetically stable attenuated virulence (deletion of purl gene), reduction of septic shock potential (deletion of msbB gene) and antibiotic susceptibility. The contrast agents that are uploaded to the attenuated bacteria strains may or may not be encapsulated by the biocompatible coating.

The magnetic nanoparticles may be uploaded to attenuated bacteria strains via incubating the bacteria with the magnetic nanoparticies. For example, experiments were conducted in which magnetic nanoparticles ranging from 80-120 nm in size were utilized. As can be seen from Table 1, the conditions of incubating the bacteria with magnetic nanoparticles varied with respect to time (30 or 120 minutes) and temperature (4° C., 24° C., or 37° C.). In these experiments, 1×10⁸ colony forming units (CFUs) of Salmonella strain BRD509 were incubated with magnetic nanoparticles in saline buffer. At the end of the incubation period, the bacterial suspension was spun down and the supernatant was aspirated. After resuspending the bacterial pellet in 1 ml saline, the bacterial suspension was subjected to a 0.45 Tesla permanent magnet for 15 minutes on the outside surface of the eppendorf tube, as shown in FIG. 10. The remaining supernatant, presumably containing bacteria without magnetic nanoparticles, was aspirated, and replaced with fresh saline. This procedure was repeated three times. Aliquots were removed from the bacterial suspension before and after each wash cycle and plated to determine the actual count of bacterial CFUs. Using this procedure, the number of bacterial CFUs remaining after four cycles of magnetic separation and washing (which most likely represents the number of bacteria actually associated with magnetic nanoparticles) was determined, and hence the percentage of bacteria associated with the magnetic nanoparticles was calculated. The results of this analysis are summarized. FIG. 10 illustrates the loss of Salmonella organisms without magnetic nanoparticles, following co-incubation with magnetic nanoparticles at 24° C. for 120 minutes, after each cycle of wash. This demonstrates that all bacteria not associated with nanoparticies are effectively removed by the third wash cycle. Furthermore, as shown in the data in Table 1, varying the incubation conditions have a clear impact on the uptake of magnetic nanoparticles by the bacteria. Incubation of magnetic nanoparticles with live Salmonella organisms at room temperature resulted in uptake of about six percent (6×10⁶), which was sufficient for the loading purpose. The fact that the association appears to be strong suggests that it is feasible to use the Salmonella organisms loaded with magnetic nanoparticles in tumor-targeting in vivo.

TABLE 1
Relative efficiency of magnetic nanoparticle uptake by Salmonella under
different incubation conditions.

| | |
|---|---|
| Incubation Conditions | Percent of MNP |

| Time (minutes) | Temperature (° C.) | loaded with bacteria |
|---|---|---|
| 30 | 37 | 3.8% |
| 120 | 37 | 4.0% |
| 30 | 24 | 4.3% |
| 120 | 24 | 6.0% |
| 30 | 4 | 1.0% |
| 120 | 4 | 5.0% |

The toxicity of the magnetic nanoparticles was investigated, and the experiments determined that the cell morphology did not change. In particular, magnetic nanoparticles were incubated with fibroblasts and Caco-2 cells lines for 24 hours to test their potential toxic effect on normal human and cancer human cells. Three different concentrations of magnetic nanoparticles were used in the experiment, and the cells were examined using light microscopy. Cell morphology of the normal human and cancer human cells remained unchanged during the entire incubation period. FIG. 11 shows the fibroblasts in the upper panel and Caco-2 cells in the lower panel. There was no toxic response observed for the bacteria incubated with magnetic nanoparticles.

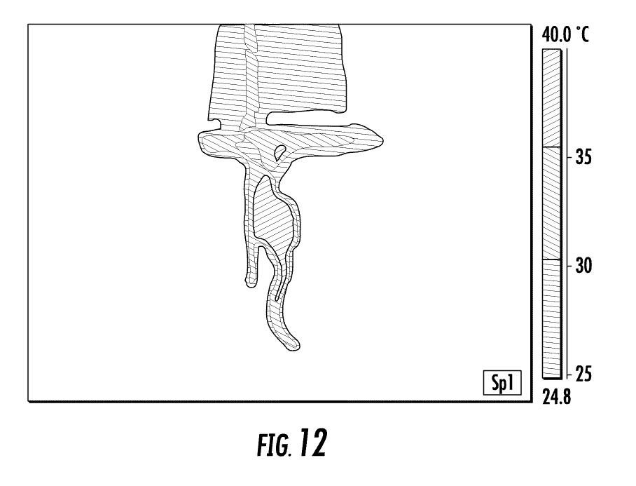

During use, the contrast agents may be placed, through injection or otherwise, into a bloodstream of a patient, such as a human being or animal, to identify tumors within the patient. In particular, a predetermined concentration of bacteria loaded with contrast agents may be placed into a bloodstream feeding a tumor. Once in the bloodstream, the bacteria seeks the tumor. Once the bacteria locates the tumor, the bacteria enters the tumor and resides therein. If no tumor is present, the attenuated bacteria strains are passed out of the patient within 24 hours of being injected into the patient. An alternating magnetic field may then be applied in proximity of the tumor location 24 hours after administering the loaded bacteria to the patient. The magnetic nanoparticles induce heating within the tumor tissue. In embodiments where the contrast agents are those having a Curie temperature less than a critical temperature of tissue at which the tissue is compromised, the magnetic nanoparticles, when subjected to the alternating magnetic field, heat up to a predetermined Curie temperature and do not increase in temperature beyond the Curie temperature, as shown in FIG. 4. A thermal sensitive device is utilized to scan the patient's body to identify the thermal signature of the patient. The contrast agents attached to the attenuated bacteria strains collect in the tumors throughout the patient and increase in temperature to temperatures greater than other parts of the patient. Thus, the location of tumors within the patient are identified by identifying high temperature spots within the patient. In particular, as shown in FIG. 12, a tumor may be seen in an image produced by a scan with a thermal sensitive device for a tumor loaded with magnetic particles that was subjected to an alternating field. The temperature contours identify the boundaries of the tumor.

In another embodiment, contrast agents having a predetermined magnetic moment profile may be placed, through injection or otherwise, into a bloodstream of a patient, such as a human being or animal, to identify tumors within the patient. The contrast agents collect in the tumor as previously set forth. After sufficient time has passed since the time of injection of the contrast agents, the patient may then be exposed to a MRI system to create images of the patient. Because the contrast agents have a distinct magnetic moment profile that correlates with specific temperatures, the one or more tumors in which the contrast agents reside in the patient's body may be easily identified.

The foregoing is provided for purposes of illustrating, explaining, and describing embodiments of this invention. Modifications and adaptations to these embodiments will be apparent to those skilled in the art and may be made without departing from the scope or spirit of this invention.

I claim:

1. A contrast agent for imaging, comprising:
at least one magnetic nanoparticle having a Curie temperature less than a critical temperature of tissue at which the tissue is compromised.

2. The contrast agent of claim 1, further comprising a biocompatible coating encapsulating the at least one magnetic nanoparticle.

3. The contrast agent of claim 1, wherein the at least one magnetic nanoparticle comprises a combination of magnetic and nonmagnetic materials.

4. The contrast agent of claim 1, wherein the at least one magnetic nanoparticle has a cross-sectional width of between five nm and one micron.

5. The contrast agent of claim 1, wherein the Curie temperature is less than about 44 degrees Celsius.

6. The contrast agent of claim 1, further comprising at least one attenuated bacteria strain attached to the at least one magnetic nanoparticle.

7. A contrast agent for imaging, comprising:
at least one magnetic nanoparticle having a distinct magnetic moment profile; wherein the at least one magnetic nanoparticle is detectable by a MRI system.

8. The contrast agent of claim 7, further comprising a biocompatible coating encapsulating the at least one magnetic nanoparticle.

9. The contrast agent of claim 7, wherein the at least one magnetic nanoparticle comprises a combination of magnetic and nonmagnetic materials.

10. The contrast agent of claim 7, wherein the at least one magnetic nanoparticle has a cross-sectional width of between five nm and one micron.

11. The contrast agent of claim 7, further comprising at least one attenuated bacteria strain attached to the at least one magnetic nanoparticle.

12. A delivery system for a contrast agent for use in imaging systems, comprising:
at least one magnetic nanoparticle having a Curie temperature less than a critical temperature of tissue at which the tissue is compromised;
wherein the at least one magnetic nanoparticle is in contact with an attenuated bacteria strain.

13. The delivery system of claim 12, further comprising a biocompatible coating encapsulating the at least one magnetic nanoparticle.

14. The delivery system of claim 12, wherein the at least one magnetic nanoparticle comprises a combination of magnetic and nonmagnetic materials.

15. The delivery system of claim 12, wherein the attenuated bacteria strain is Salmonella.

16. The delivery system of claim 12, wherein the Curie temperature is less than about 44 degrees Celsius.

17. A delivery system for a contrast agent for use in imaging systems, comprising:
at least one magnetic nanoparticle having a distinct magnetic moment profile;
wherein the at least one magnetic nanoparticle is in contact with an attenuated bacteria strain.

18. The delivery system of claim 17, further comprising a biocompatible coating encapsulating the at least one magnetic nanoparticle.

19. The delivery system of claim 17, wherein the at least one magnetic nanoparticle comprises a combination of magnetic and nonmagnetic materials.

20. The delivery system of claim 17, wherein the attenuated bacteria strain is Salmonella.
